SEQUENCE LISTING (1 sequence)

SEQ ID NO: 1 - protein, 18 aa, Artificial sequence
  histone H3 tail peptide
APTKQTARKSTGGKAPRK

(19) United States
(12) Patent Application Publication
(10) Pub. No.: US 2014/0065619 A1
(43) Pub. Date: Mar. 6, 2014

(54) MARKER SET OF HIF-1alpha, HDAC3 AND WDR5 FOR PREDICTING METASTASIS AND SURVIVAL OF CANCERS

(71) Applicant: Kou-Juey WU, Taipei City (TW)

(72) Inventor: Kou-Juey WU, Taipei City (TW)

(21) Appl. No.: 13/598,477

(22) Filed: Aug. 29, 2012

(51) Int. Cl. C12Q 1/68; G01N 33/68

(52) U.S. Cl. CPC C12Q 1/6886; G01N 33/6893
USPC 435/6.12; 436/501

(57) ABSTRACT

In the present invention, the mechanisms to coordinately regulate EMT marker genes during EMT and the interplay between these chromatin modifiers were examined. According to the experimental results, a set of marker consisting of HIF-1α, HDAC3, and WDR5 is provided to predict prognosis or overall survival of cancer patients. By determining if the set markers are co-expressed in a biological sample, it can reach relatively higher predictability of prognosis situation or overall survival as compared with the current markers.

### FIELD OF THE INVENTION

The present invention relates to a method of predicting metastasis and survival of cancers, especially a head and neck cancer or oral cancer. The method comprises determining the co-expression of a set of three: diagnostic markers consisting of HIF-1α, HDAC3, and WDR5.

### BACKGROUND OF THE INVENTION

Head and neck cancer refers to a group of biologically similar cancers that start in the lip, oral cavity (mouth), nasal cavity (inside the nose), paranasal sinuses, pharynx, and larynx. 90% of head and neck cancers are squamous cell carcinomas (SCCHN), originating from the mucosal lining (epithelium) of these regions. Head and neck cancers often spread to the lymph nodes of the neck, and this is often the first (and sometimes only) sign of the disease at the time of diagnosis. Head and neck cancer is strongly associated with certain environmental and lifestyle risk factors, including tobacco smoking, alcohol consumption. UV light, particular chemicals used in certain workplaces, and certain strains of viruses, such as human papillomayirus (Ridge J A, et al. in Pazdur R (Eds) Cancer Management: A Multidisciplinary Approach. 11 ed. 2008). These cancers are frequently aggressive in their biologic behavior; patients with these types of cancer are at a higher risk of developing, another cancer in the head and neck area. Head and neck cancer is highly curable if detected early, usually with some form of surgery although chemotherapy and radiation therapy may also play an important role.

Although early-stage head and neck cancers (especially laryngeal and oral cavity) have high cure rates, up to 50% of head and neck cancer patients present with advanced disease (Gourin C. Podolsky R. Laryngoscope 116 (7): 1093-106, 2006). Cure rates decrease in locally advanced cases, whose probability of cure is inversely related to tumor size and even more so to the extent of regional node involvement. Consensus panels in America (AJCC) and Europe (UICC) have established, staging systems for head and neck squamous cancers. These staging systems attempt to standardize clinical trial criteria for research studies, and attempt to define prognostic categories of disease. Squamous cell cancers of the head and neck are staged according to the TNM classification system, where T is the size and configuration of the tumor. N is the presence or absence of lymph node metastases, and M is the presence or absence of distant metastases.

Epithelial-mesenchymal transition (EMT) is fundamental for embryonic development and important for organ formation and differentiation (Thiery, J P et al. Cell 139: 871-890, 2009: Yang, J and Weinberg, R A. Dev. Cell 14: 818-829, 2008). In cancer metastasis, different EMT regulators such as Snail, Twist1, SIP1, etc. are shown to mediate EMT and metastasis under the signaling of hypoxia/HIF-1, Wnt, Notch, TGF-β, etc (Peinado. H et al. Nat. Rev. Cancer 7: 415-428, 2007; Thiery, J P et al., 2009; Yang. 1, and Weinberg, R A, 2008; Yang, M. H., and Wu, K J. Cell Cycle 7: 2090-2096, 2008).

The current markers used to predict the prognosis or overall survival of cancer patients usually did not have high predictive value or were considered controversial due to conflicting reports (Clin. Cancer Res. 12: 507-515, 2006). There is no useful markers (or marker set) that combine HIF-1α with the markers of chromatin modifiers to significantly predict the metastasis potential and overall survival of head and neck cancer patients (Thomas, G R et al. Int. Exp. Pathol. 86: 347-363, 2005: Massano, J et al. Oral. Surg. Oral. Med. Oral. Pathol. Oral. Radio. Endod. 102: 67-76, 2006; Chen, Z G, Curr. Cancer Drug Targets 7: 613-622, 2007; Chang, S S & Califano, J. J. Surg Oncol. 97: 640-643. 2008: Hunt J L. Arch. Pathol. Lab. Med. 135: 602-609, 2011). In this invention, the mechanisms to coordinately regulate EMT marker genes during EMT and the interplay between these chromatin modifiers were examined.

### SUMMARY OF THE INVENTION

This invention is based on the novel discovery that marker set of HIF-1α, HDAC3. and WDR5 correctly predicted the metastatic potential of head and neck cancer patients. The patients with co-expression of these three markers were considered to have the worst prognosis and will require extensive therapeutic intervention.

In one aspect, the present invention features a method of predicting metastasis and survival of cancers. The method is characterized by determining the expression levels and performing combined statistical analysis of a marker set of HIF-1α, HDAC3, and WDR5.

The predicting method comprises steps of obtaining a biological sample from the cancer patient; determining if all the three diagnostic markers consisting of HIF-1α, HDAC3, and WDR5 are co-expressed in the biological sample; and comparing the diagnostic markers determined to be co-expressed in the biological sample with their absence in a control sample; wherein the co-expression of the three diagnostic markers in the biological sample being indicators for the metastasis and overall survival of the cancer patient.

To further explanation, the control sample is a group which does not co-express all the three diagnostic markers consisting of HIF-1α, HDAC3 and WDR5.

Preferably, the cancer is head and neck squamous cell carcinoma (HNSCC). Preferably, the cancer is oral cancer.

Preferably, the co-expression of the diagnostic markers is determined by immunohistochemistry (IHC) analysis.

Preferably, the co-expression of the diagnostic markers is determined by measuring the mRNA levels of the diagnostic markers. More preferably, the mRNA levels of the diagnostic markers are quantified by real time PCR (qRT-PCR) analysis.

In another aspect, this invention features a set of diagnostic markers consisting of HIF-1α, HDAC3 and WDR5, wherein the co-expression of all these three diagnostic markers can be served as indicators for predicting the metastasis and poor overall survival of cancer patients with higher probability.

Preferably, the patients who co-express all the three diagnostic markers consisting of HIF-1α, HDAC3 and WDR5 were predicted to have high probability of metastasis. The co-expression of HIF-1α, HDAC3 and WDR5 indicates having more than 80% probability of metastasis in cancer patients. More preferably, the co-expression of HIF-1α, HDAC3 and WDR5 indicates having more than 90% probability of metastasis in HNSCC cancer patients.

Preferably, the patients who co-express all the three diagnostic markers consisting of HIF-1α, HDAC3 and WDR5 were predicted to have high probability of metastasis. The co-expression of HIF-1α, HDAC3 and WDR5 indicates having more than 50% probability of poor overall survival in cancer patients.

Other features or advantages of the present invention will be apparent from the following drawings and detailed description of several embodiments, and also from the appending claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the direct regulation of HDAC3 by HIF-1α. (A) schematic representation of the HDAC3 promoter region and the reporter constructs used in HIF-1α transfection experiments. The constructs contained wild type (pXP2-HDAC3-HRE) or mutated (pXP2-HDAC3-mut) HRE located −37 to −33 bp upstream of the transcription start site of HDAC3. (B) Luciferase activity of pXP2-HDAC3-HRE or pXP2-HDAC3-mut after co-transfection of different expression constructs under normoxia or hypoxia. The luciferase activity/β-galactosidase of 293T cells co-transfected with pXP2-HDAC3/pcDNA3 control vector under normoxia was applied as the baseline control. The asterisk (*) indicates statistical significance (P<0.05) between experimental and control transfections.

FIG. 2 shows results of Chromatin immunoprecipitation (ChIP) analysis. (A) Chromatin was incubated with IgG control or anti-HIF-1α antibody. (B) Chromatin was incubated without antibody, with anti-c-MYC antibody, or with anti-HA/HIF-1α antibodies. The 253-bp fragment contains the HRE; whereas the 202-bp fragment does not contain any HRE in the HDAC3 promoter. The 252-bp fragment contains the HRE in the VEGF promoter. Input, 2% of total input lysate.

FIG. 3 is a diagram showing Knockdown of HDAC3 in FADU-HIF1α(ΔODD) or HI299 clones reverts EMT and metastasis. (A) Western blot analysis of HIF-1α(ΔODD), HDAC3, epithelial and mesenchymal markers FADU-HIF1α(ΔODD)-HDAC3-si vs. FADU-HIF1α(ΔODD) control clones. (B) Fold change of migration and invasion activity of FADU-cDNA3, FADU-HIF1α(ΔODD) and FADU-HIF1α(ΔODD)-HDAC3-si clones. The FADU-HIF1α(ΔODD)-1 clone was selected as the control group. (C) In vivo metastatic ability of FADU-cDNA3, FADU-HIF1α(ΔODD) and FADU-HIF1α-HDAC3-si clones as assayed by tail vein injection (open bars) or orthotopic implantation (closed bars) methods. The FADU-HIF1α(ΔODD)-1 clone was selected as the control group. (D) EMT marker changes in HI299-control vs. HI299-HDAC3-si clones. HI299-top3α-si clone was also used as a control. (E) Fold change of migration and invasion activity of HI299-control vs. HI299-HDAC3-si clones HI299-control clone was used as a control. (F) In vivo metastatic ability of HI299 control and HI299-HDAC3-si clones as assayed by tail vein injection methods. HI299-control clone was used as a control. The asterisk (*) indicates statistical significance (P<0.05) between experimental and control clones. Error bars indicate standard deviations (s.d.) of quadruple measurement of migration/invasion activity (B, F) or metastastic tumor nodules in mice experiments (n=6)(C, F).

FIG. 4 shows inhibition of in z to migration:/invasion activity in FADU-HDAC3-si or MCF7-HDAC3-si clones vs. the FADU or MCF-7 control clone (under normoxia (N) or hypoxia (H)). The asterisk (*) indicates statistical significance (P<0.05) between experimental and control clones (i.e. FADU or MCF-7 control clones). Normoxic samples of each clone were used as controls vs. their hypoxic counterparts. Error bars indicate standard deviations (s.d.) of quadruple measurement of migration/invasion activity.

FIG. 5 is result of sequential qChIP assays showing (A) the recruitment of WDR5 by HDAC3 on the N-caherin/vimentin gene promoters in MCF-7 clones under hypoxia but not in MCF7-HDAC3-si clones under hypoxia; and (B) the absence of WDR5 binding on the N-cadherin or vimentin promoter in FADU-HDAC3-si or MCF7-HDAC3-si clones under hypoxia. α-HDAC3, α-WDR5: antibody against HDAC3 or WDR5. IgG was used as a negative control N: normoxia; H: hypoxia. The asterisk (*) indicates statistical significance (P<0.05) between experimental and control clones.

FIG. 6 is result of co-immunoprecipitation assay showing that the antibody against Twist1 pulled down HDAC3 and WDR5 (A); and that the antibody against WDR5 pulled down Twist1 (B). IgG was used as a negative control antibody. WCE: whole cell extracts.

FIG. 7 is result of HMT assays showing the increase in H3K4-specific HMT activity in two cell lines under hypoxia. Peptides used in the assay were described in Supplemental Experimental Procedures. α-HDAC3: antibody against HDAC3. The assays were performed in the presence of DNase.

FIG. 8 shows co-expression of HIF-1α, HDAC3 and WDR5 in HNSCC patients indicating a shorter metastasis-free period and a worse survival. (A) Immunohistochemistry o staining of a, HIF-1α, HDAC3 and WDR5 in a representative head and neck squamous cell carcinoma (HNSCC) case with co-expression of HIF-1α/HDAC3/WDR5. N, normal epithelium; T: tumor tissues. The red arrows indicate the nuclear expression of HIF-1α, HDAC3 or WDR5. The scale bars represents 100 μm. (B) Comparison of metastasis-free period in HNSCC cases with or without HIF-1α/HDAC3/WDR5 co-expression. (C) Comparison of overall survival in HNSCC cases with or without HIF-1α/HDAC3/WDR5 co-expression.

FIG. 9 shows co-expression of HIF-1α, HDAC3 and WDR5 in oral cancer cases indicating a worse survival. Overall survival in oral cancer patients with HIF-1α/HDAC3/WDR5 co-expression (n−15) was significantly worse than non-coexpression patients (n=57) (P−0.024) as categorized by mRNA levels. The log-rank test was used to compare between groups.

### DETAILED DESCRIPTION OF THE INVENTION

The specific example below is to he construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety. Further, any mechanism proposed below does not in any way restrict the scope of the claimed invention.

### EXAMPLE

### Direct Activation of HDAC3 by HIF-1α

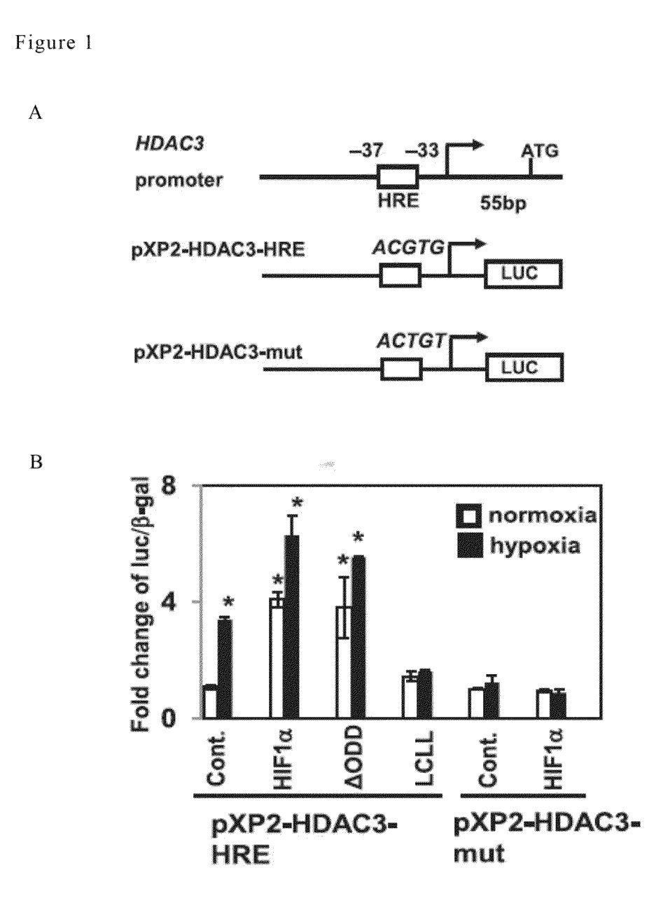

To determine whether HDAC3 is directly regulated by HIF-1α, the function of a putative hypoxia response element (HRE) in the proximal promoter of the HDAC3 gene was tested. The HDAC3 promoter region and the reporter constructs used in HIF-1α transfection experiments are showed in FIG. 1A.

A 3.5 to 4 fold increase in the HDAC3 promoter activity was observed after hypoxia or transient transfection with either wild type HIF-1α or HIF-1α(ΔODD) vector (a constitutively active mutant with deletion of the oxygen degradation domain; Huang, L E et al. Proc. Natl. Acad. Sci. USA 95:7987-7992, 1998). A further increase in the promoter activity (˜6.5 fold) was observed in cells undergoing hypoxia and overexpressing HIF-1α. The inactive HIF-1α mutant (HIF-1α(LCLL)) (Huang, 1998) failed to activate the HDAC3 promoter and site-directed mutagenesis of the putative HRE in the HDAC3 promoter prevented activation under hypoxia or HIF-1α overexpression (FIG. 1B). Increased HIF-1 binding was observed after incubation of nuclear extracts from hypoxic cells with the HRE-containing oligonucleotide from the HDAC3 promoter using electrophoretic mobility shift assays (EMSAs), and a supershifted band was detected after adding either an anti-HIF-1α or anti-HIF-1β-specific antibody to the nuclear extracts of hypoxic cells. Competition of HIF-1 binding by unlabelled oligonucleotide containing HRE abolished the HIF-1-shifted band, and probes containing mutated HRE were not shifted by HIF-1.

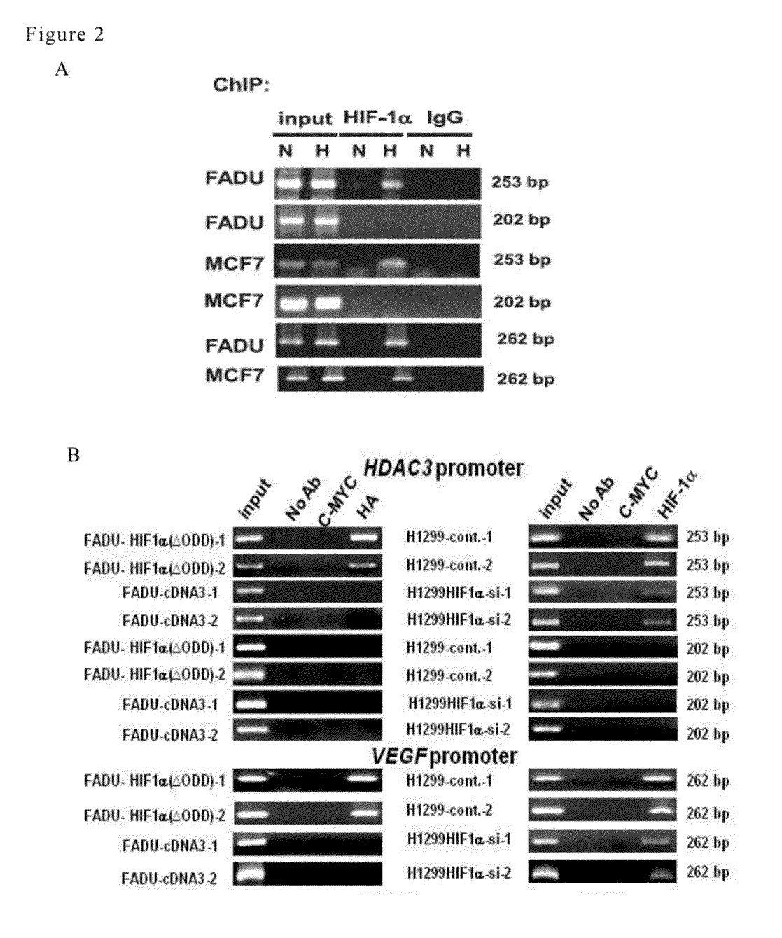

Chromatin immunoprecipitation (ChIP) assays showed that HIF-1α bound to the HDAC3 promoter containing the HRE (253 bp) in hypoxic FADU or MCF-7 cells but not in normoxic FADU or MCF-7 cells (FIG. 2). The binding was also detected in the FADU-HIF1α(ΔODD) sample but not in the FADU-cDNA3 sample; whereas knockdown of HIF-1α in HI299 cells attenuated the binding of HIF-1 to the HRE (HI299-control vs. HI299-HIF-1α-si; Figure SIF. upper panels). Control experiments showed that HIF-1 bound to the HRE in the VEGF promoter (262 bp) (FIG. 2A, lanes 5 & 6 and FIG. 2B, lower panels). These results demonstrate that HIF-1α activates HDAC3 expression directly by binding to the HRE m the HDAC3 promoter.

### HDAC3is Essential for Hypoxia/HIF-1α Induced EMT and Metastasis

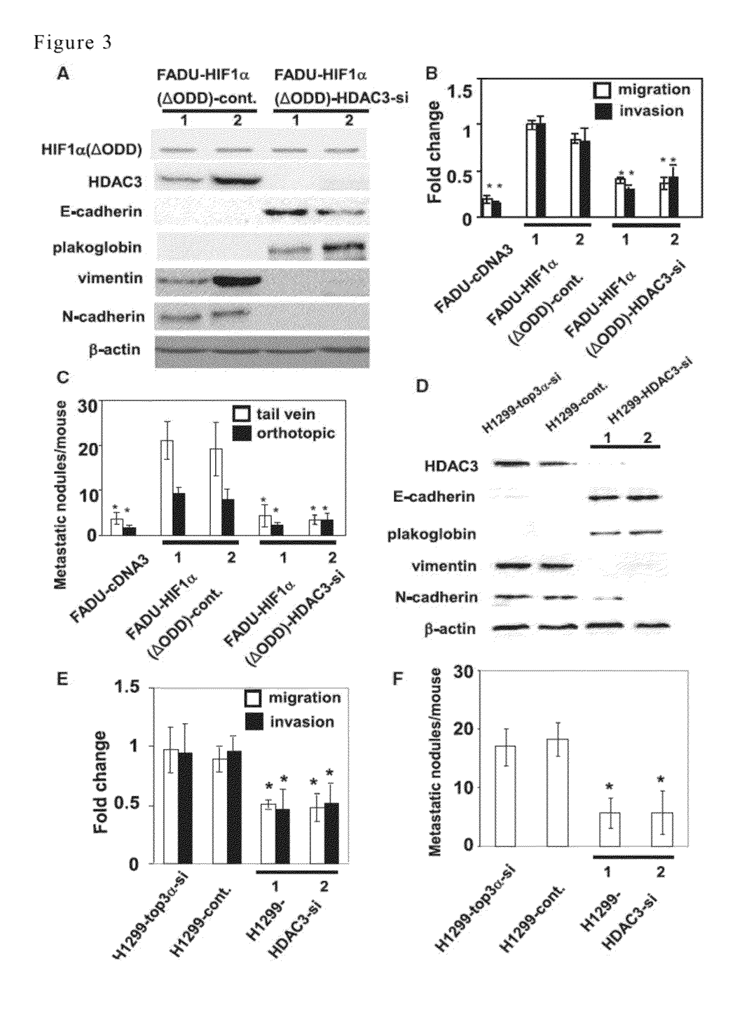

To determine whether HDAC3 is essential in HIF-1α- and hypoxia-mediated EMT and metastasis, siRNA-mediated repression of HDAC3 was performed in FADU-HIF1α(ΔODD) clones. Repression of HDAC3 in FADU-HIF1α(ΔODD)-HDAC3-si clones caused the shift in expression of mesenchymal markers vimentin N-cadherin) to epithelial markers (E-cadherin, plakoglobin), compared with the control FADU-HIF1α (ΔODD) clones (FIG. 3A). Induction of metastatic phenotypes caused by HIF-1α(ΔODD) overexpression was abolished by repression of HDAC3 as shown by migration, invasion, tail vein injections and orthotopic implantation o assays (FIGS. 3B and 3C). Similar results were observed in HI299 clones with HDAC3 repression (FIGS. 3D-3F). HDAC3 overexpression also induced EMT and metastatic phenotypes in FADU clones. The enzymatic activity of HDAC3 was required for the induction of EMT since overexpression of an inactive HDAC3 mutant (Y298F) did not induce EMT. An in vitro HDAC3 deacetylase assay further showed that wild type HDAC3, but not the HDAC3(Y 298F) mutant, deacetvlated the H3K4Ac peptide.

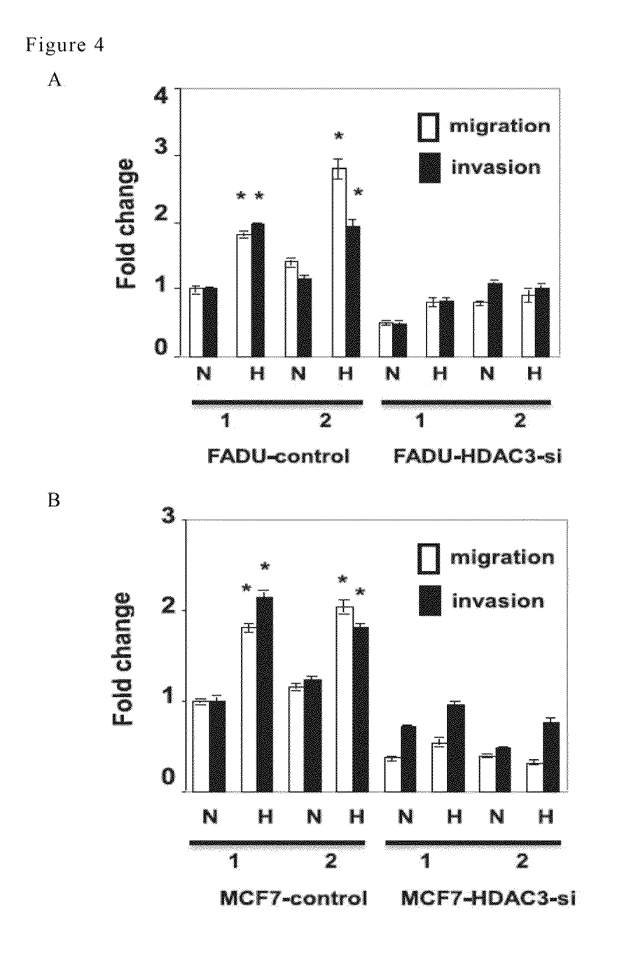

Knockdown of endogenous HDAC3 caused a complete or significant loss of EMT and inhibition of migration and invasion activity in FADU or MCF-7 clones under hypoxia even in the presence of Snail and Twist1, suggesting the critical role of HDAC3 to facilitate the regulation of EMT by different EMT regulators (FIGS. 4A and 4B). The morphology of different clones undergoing EMT was shown and the status of epithelial or mesenchymal morphology responded to HDAC3 modulation. Knockdown of endogenous HDAC3 increased apoptosis and decreased cell proliferation/growth in FADU or MCF-7 clones, which were consistent with the reported role of HDAC3 in apoptosis and cell proliferation (Narita, N et al. Oncogene 24: 7346-7354, 2005; Spurling, C C et al. Mol. Carcinog. 47: 137-147, 2008; Trivedi, C M et al, J. Biol. Chem. 283: 26484-26489, 2008). Taken together, these results demonstrate the essential and specific role of HDAC3 in the induction of EMT and metastatic phenotypes caused by hypoxia or HIF-1α overexpression.

### HDAC3 Recruits WDR5/HMT Complex to Increase H3K4-Specific Historic Methyltransferase Activity

To confirm the recruitment of WDR5 by HDAC3 on the N-cadherin and vimentin promoters during, hypoxia-induced EMT, sequential qChIP assays using the anti-HDAC3 antibody followed by the anti-WDR5 antibody were performed. For qChIP assay, DNA samples were quantified by the SYBR® Green assay using SYBR® Green PCR Master Mix (Applied Biosystems) with specific primer. Data were analyzed by the CT method and plotted as % input DNA. qChIP values were calculated by the following formula: % input recovery=[100/(input fold dilution/bound fold dilution)×2^{(input CT-bound CT)}. In sequential ChIP experiment, cells were crosslinked by incubation at RT with 1% formaldehyde for 15 min and stopped the reaction by adding 1 M glycine to final concentration of 0.125 M. The fixed cells were harvested in 5 ml of SDS buffer (50 mM Tris, pH 8.0, 0.5% SDS, 100 mM NaCl, 5 mM EDTA, and protease inhibitors). After centrifugation, cell pellets were suspended in 2 ml of IP buffer (100 mM Tris, pH 8.6, 0.3% SDS, 1.7% Triton X-100, 5 mM EDTA). After sonication, the lysates were incubated with protein A beads (50% protein A beads slurry) conjugating antibodies specific for different antibodies or IgG control. The percentage of IgG control pull down consistently reached below 0.02% of input.

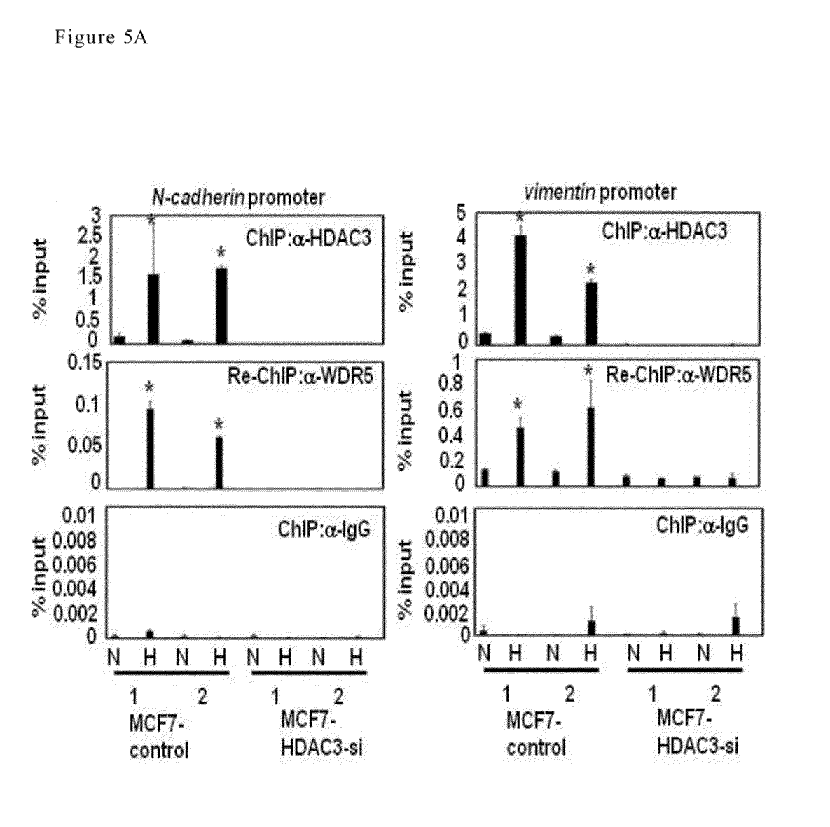

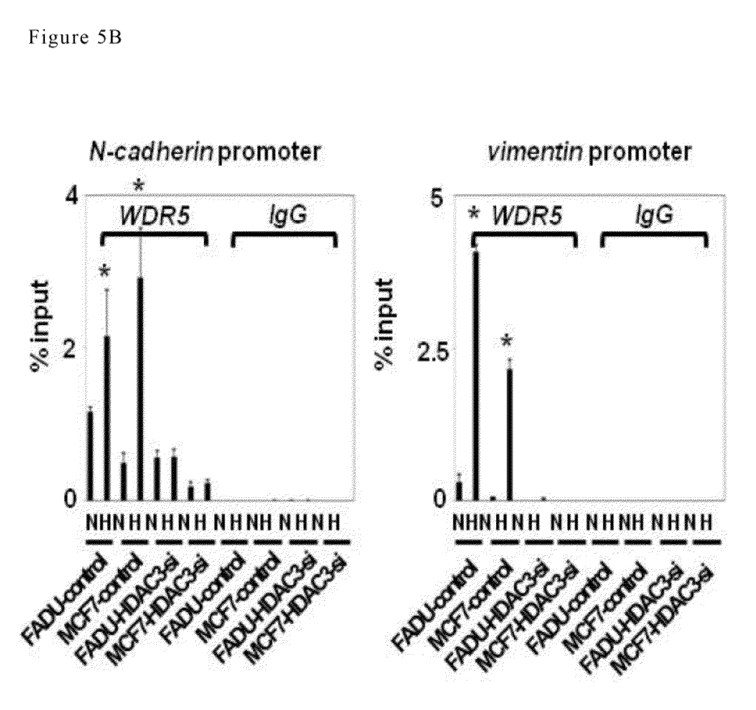

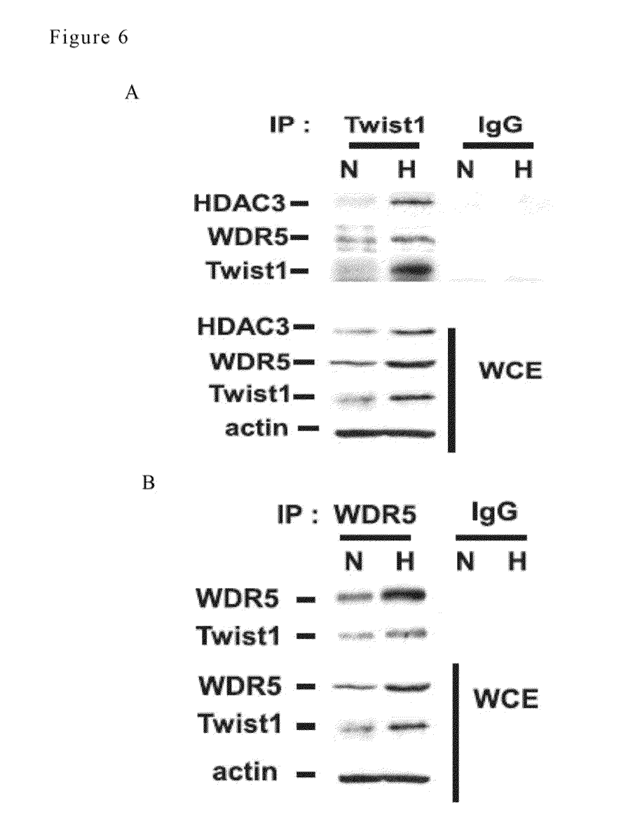

WDR5 was pulled down in the anti-HDAC3 ChIP-immunoprecipitants from hypoxic cells, but not from the HDAC3-knockdown clones under hypoxia (FIGS. 5A and 5B). qChIP experiments using the anti-WDR5 antibody in control vs. HDAC3 knockdown clones (nonnoxia or hypoxia) showed the absence of WDR5 binding to the mesenchymal gene promoters in HDAC3 knockdown clones (FIG. 5B). Co-immunoprecipitation assays showed that anti-Twist1 antibody pulled down HDAC3 and WDR5 (FIG. 6A) and the anti-WDR5 antibody pulled down Twist1 (FIG. 6B).

Finally, a HMT activity assay was performed using the anti-HDAC3 antibody-immunoprecipitants to test the ability of HDAC3 to increase HMT activity under hypoxia. Briefly, cell lysates were incubated with the anti-HDAC3 antibody or control IgG followed by reacting; in a reaction mixture containing. Cells were lysed in HMT lysis buffer (10% glycerol. 0.1% NP-40, 1 mM DTT and 1 mM EDTA) at 4° C. for 1 h. After centrifugation, the cleared lysate was subjected to immunoprecipitation with anti-HDAC3 antibody and control IgG. The immunoprecipitated samples were incubated at 30° C. for 60 min in HMT reaction buffer (50 mM Tris-HCl (pH 8.5), 5 mM MgCl₂, 4 mM DTT, 1 μM ³H-labeled S-adenosyl-L-methionine [³H]SAM and 10 μg, histone H3 tail peptides (Himalaya Biotech. Hsin-Dien, Taipei County, Taiwan)) containing the unmodified peptide (ARTKQTARKSTGGKAPRK), or peptide with K4 position modified (me1: monomethylation, me2: dimethylation, or R: replacement with arginine). Peptides were resuspended in PBS at 2 mg/ml. The reaction was stopped by adding SDS sample buffer and samples were subjected to SDS-PAGE. The final products were detected by radiography after exposure to films for 3 weeks.

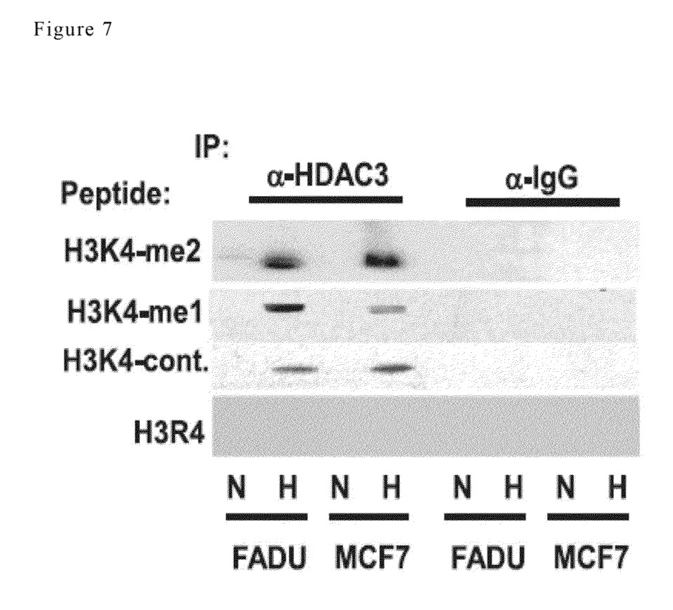

The results as described in FIG. 7 showed that histone H3 tail methyltransferase activity increased in anti-HDAC3-immunoprecipitants from two cell lines under hypoxia, compared with the immunoprecipitants from these cell lines under normoxia or control IgG-immunoprecipitants. In addition, the increased HMT activity methylated unmodified H3K4, H3K4me1 (monomethylation), and H3K4me2 (dimethylation) peptides, but not the control H3R4 peptide, demonstrating the increased H3K4-specific HMT activity in the anti-HDAC3-immunoprecipitants under hypoxia. These results suggest that HDAC3 recruits the WDR5/HMT complex to increase H3K4-specific HMT activity in hypoxic cells.

Co-Expression of HIF-1α, WDR5, and HDAC3 Correlates with Metastasis and Predicts a Worse Prognosis' of Head and Neck Squamous Cell Carcinoma (HNSCC) Patients

Tumors with increased HIF-1 activity are more likely to develop metastasis and correlate with poor survival (Gupta, G P, and Massague, J. Cell 127: 679-695, 2006; Harris, A L, Nat. Rev. Cancer 2: 38-47, 2002; Semenza, G L, Trends Mol. Med. 8(4 Suppl), S62-67, 2002). To investigate whether HDAC3 activation by HIF-1 indeed occurs in human cancers and to evaluate the prognostic significance of HIF-1α/HDAC3/WDR5 co-expression, tissue-microarray immunohistochemistry analysis of HIF-1α, HDAC3 and WDR5 expression was performed in 88 sets of HNSCC samples.

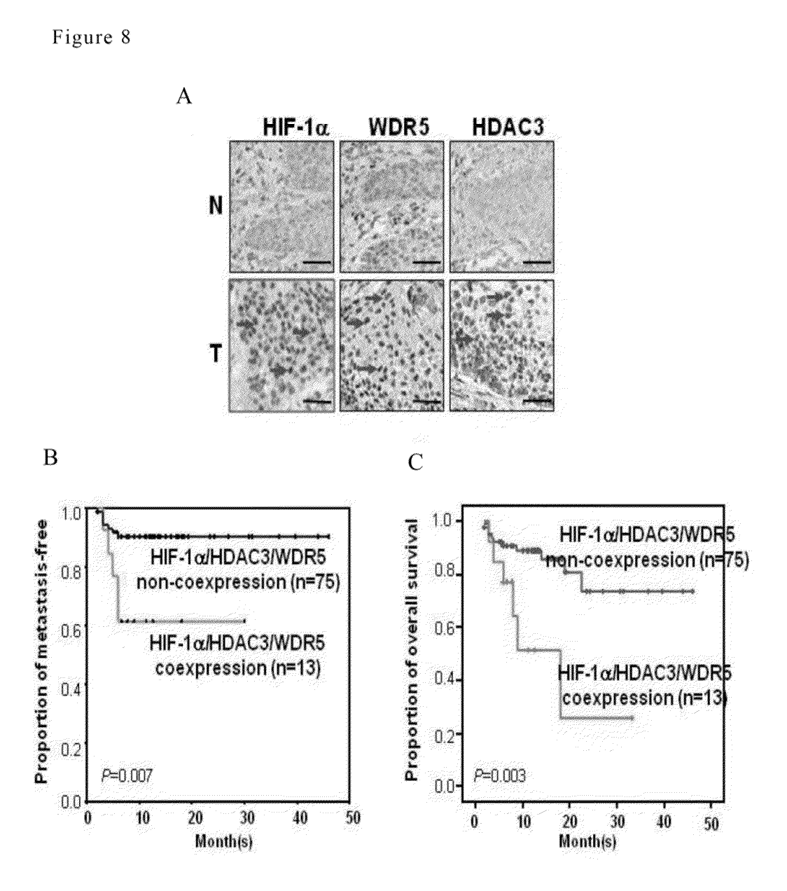

Eighty-eight HNSCC patients who underwent treatment at Taipei Mackay Memorial Hospital and Taipei Veterans General Hospital between January 2001 and December 2004 were retrospectively analyzed. This study has been approved by the institutional. Review Board of Taipei Veterans General Hospital. The clinical characteristics of 88 HNSCC patients are illustrated in Table 1. Immunohistochemistry staining of samples from a representative case with co-expression of HIF-1α/HDAC3/WDR5 are shown in FIG. 8A.

TABLE 1
Characteristics and univariate survival analysis of 88 HNSCC cases

| | | Median OS | |
|---|---|---|---|
| Variables | Case No. | (months) | P |
| Age | | | 0.543 |
| <50 | 36 | —* | |
| ≧50 | 52 | — | |
| Gender | | | 0.960 |
| Male | 83 | — | |
| Female | 5 | — | |
| T stage | | | 0.043 |
| 1~2 | 42 | — | |
| 3~4 | 46 | 25.5 | |
| N stage | | | <0.001 |
| 0 | 67 | — | |
| 1-3 | 21 | 18.0 | |
| HIF-1α overexpression | | | 0.011 |
| Yes | 40 | — | |
| No | 48 | 29.0 | |
| HDAC3 overexpression | | | 0.198 |
| Yes | 46 | — | |
| No | 42 | — | |
| WDR5 overexpression | | | 0.130 |
| Yes | 23 | — | |
| No | 65 | — | |

Primary tumor samples and the corresponding non-cancerous matched tissue were obtained during surgery. A high-density tissue microarray (TMA) was constructed using formalin-fixed, paraffin-embedded specimens of HNSCC patient samples as previously described (Kononen, J et al. Nat. Med. 4: 844-847, 1998; Yang, M H et al, Oncogene 26: 1459-1467, 2007), and constructed by Dr. Kuo-Wei Chang (Inst. of Oral Biology, National Yang-Ming Univ.).

### Statistical Analysis

The independent Student's (-test was used to compare the continuous variables between two groups, and the χ² test was applied for comparison of dichotomous variables. The Kaplan-Meier estimate was used for metastasis-free and overall survival analysis, and the log-rank test was s used to compare the difference between groups in univariate analysis. The Cox's proportional hazards model was applied in multivariate survival analysis to test independent prognostic factors. Only variables with P value less than 0.1 after the univariate analysis were entered into the multivariate analysis. The control groups of all the statistical analyses were usually the first groups in the panels unless specified otherwise in the figure legends. The level of statistical significance was set at 0.05 for all tests.

The sample processing and IHC procedure for determining the immunoreactivity of HIF-1α, HDAC3 and WDR5 were described (Weichert, W et al. Clin. Cancer Res. 14: 1669-1677, 2008; Yang et al., 2007). Briefly, 6-μm thick sections of tumor tissue were cut, from the frozen specimens for immunohistochemistry (IHC) analysis. The samples were fixed in acetone, air-dried, and subsequently bathed in Tris buffered saline (TBS) solution (pH 7.6). The endogenous peroxidase activity was blocked with 3% hydrogen peroxide. After reacting with a biotinylated secondary antibody for 30 minutes, antigen-antibody reactions were visualized using streptavidin-horseradish peroxidase conjugate (DAKO LSAB kit DAKO, Los Angeles, Calif.), with 3-amino-9-ethylcarbazole as the chromogen. All slides were counterstained with hematoxylin. For these markers, we defined the IHC result as a positive one only if the nuclear expression of the target protein was identified in ≧50% of tumor cells.

TABLE 2
Correlation of the IHC expression of HDAC3 and HIF-
1α, WDR5 in primary tumor samples of 88 HNSCC cases

HDAC3

| | | Negative | Positive | P |
|---|---|---|---|---|
| HIF-1α | Negative | 28 | 20 | 0.030 |
| | Positive | 14 | 26 | |
| WDR5 | Negative | 38 | 27 | 0.001 |
| | Positive | 4 | 19 | |

As shown in Table 2, tumors with increased HIF-1α expression significantly correlated with HDAC3 overexpression (P=0.030), and the expression level of HDAC3 was also associated with WDR5 (P=0.001). Prognostic prediction analysis showed that co-expression of HIF-1α/HDAC3/WDR5 had a significantly shorter metastasis-free period and a significantly worse outcome than did non-co-expression cases (P=0.003; FIGS. 8B and 8C). The prognostic effect of HIF-1α/HDAC3/WDR5 co-expression was independent of other prognostic markers (advanced T stage, N stage) (P=0.029; Table 3).

TABLE 3
Multivariate survival analysis of 88 HNSCC cases

OS

| | Hazard ratio | |
|---|---|---|
| Variables | (95% CI) | P |
| T stage | 2.01 | 0.212 |
| (T3~4 vs. T1~2) | (0.67-6.00) | |
| N stage | 3.30 | 0.024 |
| (N1-3 vs. N0) | (1.17-9.30) | |
| HIF-1α/HDAC3/WDR5 | 3.15 | 0.029 |
| co-expression | (1.13-8.77) | |
| (Yes vs. No) | | |

Abbreviations: OS, overall survival; CI, confidence interval.

As conclusion, it was evident, that the activation of HDAC3 and/or WDR5 by HIF-1α indeed occurs in HNSCC cancers, and the expression profile of HIF-1α, HDAC3 and WDR5 could be used as an evaluative indicator for the prognostic significance of cancer patients. In another word, co-expression of HIF-1α, HDAC3 and WDR5 could be used as a valuable marker to predict prognosis or overall survival in HNSCC patients.

### Co-Expression of HIF-1α, HDAC3 and WDR5 in Oral Cancer Cases Indicates a Worse Survival.

For the 72 oral cancer patient samples used in qRT-PCR analysis, the samples were collected at National Taiwan University Hospital (NTUH) and the study was approved by the IRB of NTUH.

TABLE 4
Characteristics and univariate survival
analysis of 72 oral cancer cases

| | | Median OS | |
|---|---|---|---|
| Variables | Case No. | (months) | P |
| Age | | | 0.325 |
| <56 | 36 | —* | |
| ≧56 | 36 | 44.2 | |
| Gender | | | 0.126 |
| Male | 65 | —* | |
| Female | 7 | —* | |
| T stage | | | 0.012 |
| 1~2 | 44 | —* | |
| 3~4 | 28 | 19.7 | |
| N stage | | | 0.044 |
| 0-1 | 48 | 44.2 | |
| 2-3 | 24 | 17.3 | |
| HIF-1α overexpression | | | 0.046 |
| Yes | 36 | 20.3 | |
| No | 36 | 44.2 | |
| HDAC3 overexpression | | | 0.193 |
| Yes | 36 | 44.2 | |
| No | 36 | —* | |
| WDR5 overexpression | | | 0.600 |
| Yes | 36 | 44.2 | |
| No | 36 | —* | |

Abbreviation: OS, overall survival
*Median survival was not reached.

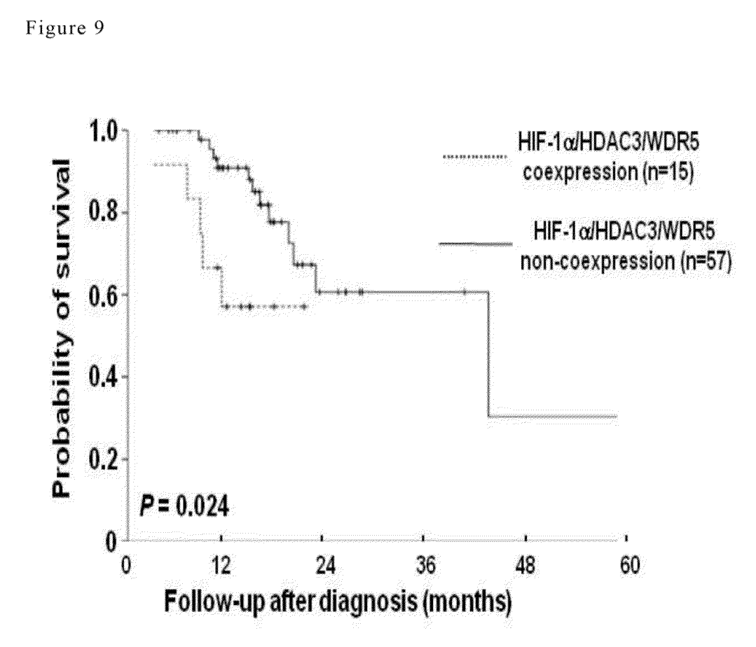

A different series of oral cancer patients were analyzed using qRT-PCR to categorize patients according to the mRNA levels, mRNA levels of HIF-1α, HDAC3 and WDR5 quantified by qRT-PCR were measured as fold change of tumor samples vs. normal tissues. For HIF-1α, the median of fold change of mRNA levels was used as a cutoff point to categorize the tumors into HIF-1α overexpression vs. HIF-1α normal levels. Similar method was used to determine the levels of HDAC3 or WDR5. The results of FIG. 9 also showed that overall survival in oral cancer patients with HIF-1α/HDAC3/WDR5 co-expression (n=15) was significantly worse than non-coexpression patients (n=57) (P=0.024) as categorized by mRNA levels. The log-rank test was used to compare between groups. That is, co-expression of HIF-1α, HDAC3 and WDR5 could be used as a valuable marker to predict prognosis or overall survival in oral cancer patients.

TABLE 5
Multivariate survival analysis of 72 oral cancer cases

OS

| | Hazard ratio | |
|---|---|---|
| Variables | (95% CI) | P |
| T stage | 3.56 | 0.016 |
| (T3~4 vs. T1~2) | (1.27-10.01) | |
| N stage | 2.79 | 0.065 |
| (N2-3 vs. N0-1) | (0.94-8.29) | |
| HIF1α/HDAC3/WDR5 | 5.34 | 0.005 |
| co-expression | (1.68-17.02) | |
| (Yes vs. No) | | |

Abbreviations: OS, overall survival; CI, confidence interval.

Collectively, the correlation analysis indicates that activation of HDAC73 by HIF-1 and associations of WDR5 with HDAC3 indeed occur in HNSCC patients and in oral cancer samples, and survival analysis supports the prognostic value of co-expression of HIF-1α/HDAC3/WDR5 in HNSCC patients and in oral cancer cases.

Based on the above, the present invention provides at least two diagnostic markers selected from the group consisting of HIF-1α, HDAC3 and WDR5 as a set of reliable markets to predict the prognosis or the overall survival of cancer patients as compared with current effective markers.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method of predicting metastasis and survival of a cancer in a cancer patient, which is characterized by determining the expression levels and performing combined statistical analysis of a diagnostic marker set consisting of HIF-1α, HDAC3, and WDR5.

2. The method of claim 1, which comprises steps of:
obtaining a biological sample from the cancer patient;
determining if all the three diagnostic markers consisting of HIF-1α, HDAC3, and WDR5 are co-expressed in the biological sample; and
comparing the diagnostic markers determined to be co-expressed in the biological sample with their absence in a control sample, the co-expression of the diagnostic markers in the biological sample being indicators for the metastasis and overall survival of the cancer patient;
wherein the control sample is a group which does not co-express all the three diagnostic markers consisting of HIF-1α, HDAC3, and WDR5.

3. The method of claim 1, wherein the co-expression of the three diagnostic markers in the biological sample indicates having more than 80% probability of metastasis in the cancer patient.

4. The method of claim 1, wherein the co-expression of the three diagnostic markers in the biological sample indicates having more than 50% probability of poor overall survival in the cancer patient.

5. The method of claim 1, wherein the cancer is head and neck squamous cell carcinoma (HNSCC).

6. The method of claim 1, wherein the cancer is oral cancer.

7. The method of claim 1, wherein the co-expression of the diagnostic markers is determined by immunohistochemistry (IHC) analysis.

8. The method of claim 1, wherein the co-expression of the diagnostic markers is determined by measuring the mRNA levels of the diagnostic markers.

9. The method of claim 1, wherein the mRNA levels of the diagnostic markers are quantified by real time PCR (qRT-PCR) analysis.
